(19) United States
(12) Patent Application Publication
(10) Pub. No.: US 2024/0228530 A1
(43) Pub. Date: Jul. 11, 2024

(54) A METHOD OF SEPARATING BISPECIFIC ANTIBODIES

(71) Applicant: Cytiva BioProcess R&D AB, Uppsala (SE)

(72) Inventors: Bengt Westerlund, Uppsala (SE); Mats Ander, Uppsala (SE)

(21) Appl. No.: 18/562,092

(22) PCT Filed: Jun. 22, 2022
(86) PCT No.: PCT/EP2022/067013

§ 371 Date: Nov. 17, 2023

(30) Foreign Application Priority Data
Jun. 28, 2021 (GB) 2109246.5

(51) Int. Cl. C07K 1/22

(52) U.S. Cl. CPC C07K 1/22; C07K 2317/31; C07K 2317/56

(57) ABSTRACT

The present invention relates to a method of separating bispecific antibodies or bispecific antibody fragments. The method comprises the steps of a) providing a feed comprising bispecific antibodies or bispecific antibody fragments; b) contacting the feed with a separation matrix having affinity ligands coupled to a support; c) optionally washing the separation resin with a washing liquid; d) applying an elution buffer to the separation resin, to elute the antibodies or antibody fragments bound to the affinity ligand; wherein in step d) a pH gradient is applied over the elution buffer, said pH gradient being from about 6 to about 2.

### TECHNICAL FIELD

The present invention relates to the field of biotechnology and a novel method for separating antibodies and antibody fragments, and in particular separation of bispecific antibodies and bispecific antibody fragments.

### BACKGROUND

Immunoglobulins represent the most prevalent biopharmaceutical products in either manufacture or development worldwide. The high commercial demand for and hence value of the therapeutic market for antibodies has led to the emphasis being placed on pharmaceutical companies to maximize the productivity of their respective monoclonal antibody (mAb) manufacturing processes whilst controlling the associated costs.

Affinity chromatography is used in most cases, as one of the key steps in the purification of immunoglobulin molecules, such as monoclonal or polyclonal antibodies. A particularly interesting class of affinity reagents is proteins capable of specific binding to invariable parts of an immunoglobulin molecule, such interaction being independent on the antigen-binding specificity of the antibody. Such reagents can be widely used for affinity chromatography recovery of immunoglobulins from different samples such as but not limited to serum or plasma preparations or cell culture derived feed stocks. An example of such a protein is staphylococcal protein A, containing domains capable of binding to the Fc and Fab portions of IgG immunoglobulins from different species. These domains are commonly denoted as the E-, D-, A-, B- and C-domains.

Staphylococcal protein A (SpA) based reagents have due to their high affinity and selectivity found a widespread use in the field of biotechnology, e.g. in affinity chromatography for capture and purification of antibodies as well as for detection or quantification. At present, SpA-based affinity medium probably is the most widely used affinity chromatography resin for isolation of monoclonal antibodies and their fragments from different samples including industrial cell culture supernatants. Accordingly, various matrices comprising protein A-ligands are commercially available, for example, in the form of native protein A (e.g. Protein A Sepharose™, Cytiva, Uppsala, Sweden) and also comprised of recombinant protein A (e.g. rProtein A Sepharose™, Cytiva). More specifically, the genetic manipulation performed in the commercial recombinant protein A product is aimed at facilitating oriented attachment thereof to a support and at increasing productivity of the ligand.

Monoclonal antibodies (mAb) are mono-specific antibodies, meaning that they bind one type of antigen. They are produced by hybridoma cells, and all mAbs produced by one hybridoma cell line are identical.

Bispecific antibodies (bsAb) are engineered antibodies more extensively being used as a tool within biotechnology. They also possess a therapeutic potential, with a large number of clinical trials ongoing. A bispecific antibody is an antibody that includes two different antigen binding sites, e.g. can bind two epitopes on the same antigen, or on different antigens. Hence, a bispecific antibody can potentially bind to two different antigens. Bispecific antibodies with defined specificities are artificial molecules, and per se not found in nature. A range of bispecific antibodies are further described in more detail in U. Brinkmann, et al. (Brinkmann. U., Kontermann, R. E., MAbs, 2017 February-March; 9(2):182-212).

### SUMMARY OF THE INVENTION

The generation of bispecific IgG molecules is difficult as the pairing of the light and heavy chains, and consequently the variable domains therein (VL; VH) may be promiscuous. The pairing of two different light and two different heavy chains may lead to a large number of mispairing, as normally only one specific asymmetric combination is wanted, and a multitude of combinations achieved will be non-functional or unwanted molecules, such as for instance monospecific homodimers. Thus, there is an increasing need for improved tools and methods to separate bispecific antibodies from monospecific antibodies, as well as separating mismatched bispecific antibodies from correctly matched bispecific antibodies. Thus, the objective of the inventors has been to find an improved method for enabling separation of bispecific antibodies accordingly.

Variable heavy class 3 (VH3) is a subclass of the variable heavy (VH) chain that has been developed further within biotechnology, as it demonstrates improved expression and stability over other heavy chain subclasses.

To solve the above-mentioned objective the inventors have developed a method that enables to separate bi-specific antibodies from monospecific antibodies, or mismatched antibodies, based on the presence of a VH3-chain. A monoclonal antibody has two identical VH-chains. A bispecific antibody can be designed to comprise two different VH-chains. This method of separation may be performed using any commercially available Protein A ligand with maintained VH3 interaction. The separation is performed by using a pH gradient when eluting bispecific antibodies from a Protein A ligand chromatography matrix.

Thus, the object above is being attained by a method of separating bispecific antibodies or bispecific antibody fragments, said method comprising the steps of:
a) providing a feed comprising bispecific antibodies or bispecific antibody fragments;
b) contacting the feed with a separation matrix having affinity ligands coupled to a support;
c) optionally washing the separation resin with a washing liquid;
d) applying an elution buffer to the separation resin, to elute the antibodies or antibody fragments bound to the affinity ligand.

In step d) a pH gradient is applied over the elution buffer. Said pH gradient is from about 6 to about 2.

The pH gradient may be from about 5.5, or from about 5.2, or from about 5.0, to about 2.0, or to about 2.5.

The affinity ligand may be a protein A ligand. The protein A ligand may comprise a native or mutated domain chosen from a group comprising of Z domain, A domain, B domain, C domain, D domain and E domain. Preferably, the protein A ligand may comprise a native or mutated Z domain or a native or mutated C domain.

The support may comprise crosslinked agarose beads. Alternatively, the support may comprise fibers with a cross-sectional diameter of 10-1000 nm, such as 200-800 nm, 200-400 nm or 300-400 nm.

The feed used in the method above may be a clarified cell culture supernatant.

The antibody fragment may be any antibody fragment comprising two different VH-chains, of which one VH-chain is VH3. The antibody fragment may be selected from a diabody, a scFv-Fc, a scFv-CH, Fab-scFv-Fc, or a scFv-zipper.

The antibody may be any antibody with two different VH-chains, of which one VH-chain is VH3.

### DRAWINGS

FIG. 1 is a schematic drawing of an antibody, such as an IgG.

FIG. 2 is a chromatogram showing the lack of separation of an antibody with one VH3-region from an antibody with two VH3-regions using the commercial product MabSelect™ SuRe from Cytiva.

FIG. 3 is a chromatogram showing the separation of an antibody with one VH3-region from an antibody with two VH3-regions using the commercial product MabSelect™ PrismA from Cytiva.

FIG. 4 is a chromatogram showing the separation of an antibody with one VH3-region from an antibody with two VH3-regions using the commercial product Amsphere™ A3 from JSR.

FIG. 5 is a chromatogram showing the separation of an antibody with one VH3-region from an antibody with two VH3-regions using the commercial product Praesto® Jetted A50 from Purolite.

### DEFINITIONS

The terms “antibody” and “immunoglobulin” may be used interchangeably herein and refers to an antigen-binding protein having a basic four-polypeptide chain structure consisting of two heavy (H) chains and two light (L) chains, said chains being stabilized by interchain disulfide bonds. Each heavy chain is comprised of a heavy chain variable region (abbreviated herein as HCVR or VH) and a heavy chain constant region (CH). The heavy chain constant region is comprised of three domains, CH1, CH2 and CH3. Each light chain is comprised of a light chain variable region (VL) and a light chain constant region. The light chain constant region is comprised of one domain, CL. The term is to be understood to include also fragments of antibodies, fusion proteins comprising antibodies or antibody fragments and conjugates comprising antibodies or antibody fragments.

The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabe and/or Fv fragments. The term “antigen-binding fragment” refers to a polypeptide fragment of an immunoglobulin or antibody that binds antigen or competes with the intact antibody from which they were derived for specific antigen binding.

The terms an “Fc-binding polypeptide”, “Fc-binding agent” and “Fc-binding protein” mean a polypeptide, molecule or protein respectively, capable of binding to the crystallizable part, the Fc-region of an antibody and includes, but is not limited to, e.g. Protein A and Protein G, or any fragment or fusion protein thereof that has maintained said binding property.

The term “Fc region” refers to a C-terminal region of an IgG antibody, in particular, the C-terminal region of the heavy chain(s) of said IgG antibody.

The term “Fv fragment” refers to the fragment variable region and contains only the two variable domains, VH and VL. The VH and VL are held together in Fv fragments by non-covalent interactions. The term “Fab fragment” refers to the fragment antigen-binding region and includes both a constant domain and the variable domains of both the heavy and light chains. The term “F(ab) fragment” refers to a fragment having one antigen-binding site. The term “(Fab′)2 fragments” refers to divalent fragments having two antigen-binding regions that are linked by disulfide bonds. Reduction of F(ab′)2 fragments produce 2 monovalent Fab′ fragments, which have a free sulfhydryl group that is useful for conjugation to other molecules.

“Variable” domains on the heavy chain may be referred to interchangeably as “heavy chain variable regions”, “heavy chain variable domains”, “VH” regions, “VH” domains or “VH” chains.

The terms “domain” and “region” are interchangeably used in the present disclosure.

The term “feed” as used herein, refers to a liquid containing at least one target substance which is sought to be purified from other substances also present. Feeds can, for example, be aqueous solutions, organic solvent systems, or aqueous/organic solvent mixtures or solutions. The source liquids are often complex mixtures or solutions containing many biological molecules (such as proteins, antibodies, hormones, and viruses), small molecules (such as salts, sugars, lipids, etc.) and even particulate matter. While a typical source liquid of biological origin may begin as an aqueous solution or suspension, it may also contain organic solvents used in earlier separation steps such as solvent precipitations, extractions, and the like. Examples of feeds that may contain valuable biological substances amenable to the purification by various embodiments of the present invention include, but are not limited to, a culture supernatant from a bioreactor, a homogenized cell suspension, plasma, plasma fractions, and milk.

As used herein, the term “solid support” refers to a non-aqueous matrix with which a target substance interacts during purification or to which an Fe binding agent can adhere. Suitable solid phase materials include, but are not limited to, glass, silica (e.g., silica gel), polysaccharides (e.g., a polysaccharide matrix) such as agarose and cellulose, organic polymers such as polyacrylamide, methylmethacrylate, and polystyrenedivinylbenzene copolymers. The solid phase can be of porous or nonporous character and can be compressible or incompressible. In various embodiments, the solid phase is a polymeric matrix or an agarose particle or bead. Preferred solid support materials will be physically and chemically resilient to the conditions employed in the purification process including pumping and cross-flow filtration, and temperatures, pH, and other aspects of the liquids employed.

“Affinity ligand” refers to a moiety that binds selectively or preferentially to a component of the source liquid through a specific interaction with a binding site of the component. In the present invention, the affinity ligand is typically immobilized to a solid phase such as a resin. Examples of affinity ligands that can be bound to the resin support to provide chromatography resins useful in the process of the present invention include, but are not limited to, Protein A, Protein G, and their analogs, which selectively bind to a protein Fc region. Methods of binding affinity ligands to solid support materials are well known in the purification art.

A “buffer” is a substance which, by its presence in solution, increases the amount of acid or alkali that must be added to cause unit change in pH. A buffered solution resists changes in pH by the action of its acid-base conjugate components. Buffered solutions for use with biological reagents are generally capable of maintaining a constant concentration of hydrogen ions such that the pH of the solution is within a physiological range. The term “physiological pH” refers to the pH of mammalian blood (i.e., 7.38 or about 7.4). Thus, a physiologic pH range is from about 7.2 to 7.6. Traditional buffer components include, but are not limited to, organic and inorganic salts, acids and bases. Exemplary buffers for use in purification of biological molecules (e.g., protein molecules) include the zwitterionic or “Good” Buffers, see e.g., Good et al. (1966) Biochemistry 5:467 and Good and Izawa (1972) Methods Enzymol. 24:62.

The “equilibration buffer” herein is a buffer used to prepare the binding reagent, solid phase, or both, for loading of the source liquid containing the target protein. The equilibration buffer is preferably isotonic and commonly has a pH in the range from about 6 to about 8. The “loading buffer” is a buffer used to load the source liquid containing the binding region containing protein and impurities onto the solid phase to which the binding agent is immobilized. Often, the equilibration and loading buffers are the same. The “elution buffer” is used to elute the binding region-containing protein from the immobilized binding agent. Preferably the elution buffer has a low pH and thereby disrupts interactions between the Fc binding agent and the protein of interest. Preferably, the low pH elution buffer has a pH in the range from about 2 to about 5, most preferably in the range from about 3 to about 4. Examples of buffers that will control the pH within this range include glycine, phosphate, acetate, citrate and ammonium buffers, as well as combinations of these. The preferred such buffers are citrate and acetate buffers, most preferably sodium citrate or sodium acetate buffers. Other elution buffers are contemplated including high pH buffers (e.g. those having a pH of 9 or more) or buffers comprising a compound or composition such as MgCl₂ (2 mM) for eluting the protein of interest.

“Wash liquid” or “wash buffer” as used herein all refer herein to the liquid used to carry away impurities from the chromatography resin to which is bound the target substance. More than one wash liquid can be employed sequentially, e.g., with the successive wash liquids having varying properties such as pH, conductivity, solvent concentration, etc., designed to dissociate and remove varying types of impurities that are non-specifically associated with the chromatography resin.

“Elution liquid” or “elution buffer” refers herein to the liquid that is used to dissociate the target substance from the chromatography resin after it has been washed with one or more wash liquids. The elution liquid acts to dissociate the target substance without denaturing it irreversibly. Typical elution liquids are well known in the chromatography art and may have higher concentrations of salts, free affinity ligands or analogs, or other substances that promote dissociation of the target substance from the chromatography resin. “Elution conditions” refers to process conditions imposed on the target substance-bound chromatography resin that dissociate the target substance from the chromatography resin, such as the contacting of the target substance-bound chromatography resin with an elution liquid or elution buffer to produce such dissociation.

As used herein, the terms “comprises”, “comprising”, “containing”, “having” and the like can have the meaning ascribed to them in U.S. Patent law and can mean “includes”, “including”, and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

### DETAILED DESCRIPTION

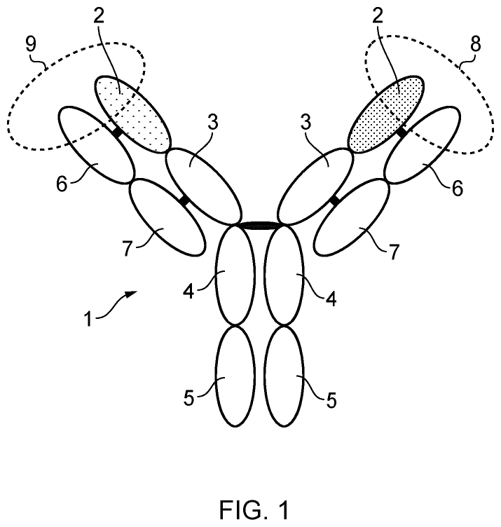

The most common antibody produced by humans is immunoglobulin G (IgG). IgG consists of four different polypeptide chains, two identical heavy chains (HC) and two identical light chains (LC). FIG. 1 schematically depicts an IgG and is referred to in the following description. The HCs both consist of one variable domain denoted VH 2 and three constant domains denoted CH1 3, CH2 4 and CH3 5, respectively. The C-terminal of the heavy chains is at the end of the CH3 domain 5. The heavy chains are held together at a hinge region which is responsible for a covalent linkage between the two chains. The two light chains are covalently attached to the heavy chains by disulfide bonds. These light chains consist of one variable domain denoted VL 6 and one constant domain denoted CL 7. The light chains together with the VH and CH1 form the antigen binding fragments (Fabs) that can bind a single antigen. A monoclonal antibody normally has two identical HC and LC. In particular, a monoclonal antibody normally has two identical VH domains 2, since it is monospecific. A bispecific antibody has two different variable regions (Fv) 8, 9 and may thus comprise two different VH domains 2, which in FIG. 1 is illustrated with different patterns for the two VH domains. 2

The part of the antibody where the two heavy chains are covalently linked is called the crystallizable fragment (Fc), and thus consists of the CH3 5 and CH2 4 domains of both heavy chains. An Fc can bind to various Fc receptors (FcR) thereby participating or mediating effector function within the immune system. Not all antibodies have a Fc region, for instance IgM lack the Fc region.

The Protein A ligands selectively bind to the Fc region of the immunoglobulins and thus allow for a highly efficient capture step. Native Protein A can still be useful in that it also binds to the VH3 region of immunoglobulins. However, native Protein A is not stable under the alkaline cleaning conditions used in bioprocessing and some alkali-stabilized Protein A variants have mutations that inhibit the VH3 interaction, see e.g. US20060194950. This document discusses the inhibition of the VH3 interaction by a G29A mutation in Protein A Fc-binding B domains, resulting in a Z domain used in the commercial product MabSelect™ SuRe.

The inventors have developed a method that enables to separate bi-specific antibodies from monospecific antibodies, or mismatched antibodies, based on the VH3-chain, using any commercially available Protein A ligand with maintained VH3 interaction. In an alternative language, the affinity ligand used in the method disclosed herein is a VH3 binding protein A ligand. The separation is performed by using a pH gradient when eluting the antibodies from a Protein A ligand chromatography resin comprising said VH3 binding protein A ligand.

The method of separating bispecific antibodies or bispecific antibody fragments according to the present invention comprises the steps of:
a) providing a feed comprising bispecific antibodies or bispecific antibody fragments;
b) contacting the feed with a separation matrix having affinity ligands coupled to a solid support;
c) optionally washing the separation resin with a washing liquid;
d) applying an elution buffer to the separation resin, to elute the antibodies or antibody fragments bound to the affinity ligand.

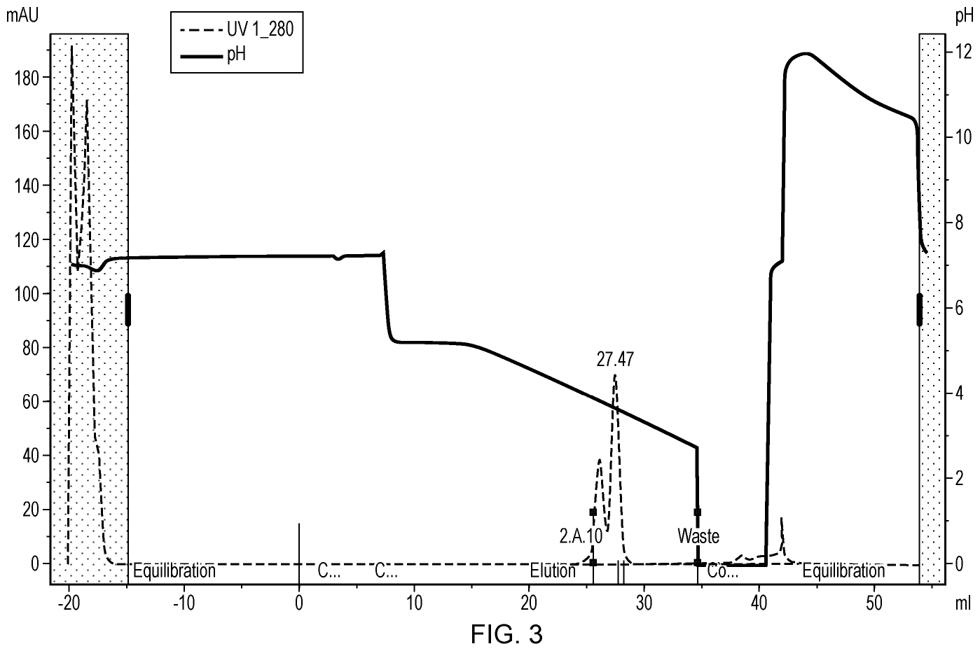

In step d) a pH gradient is applied over the elution buffer, said pH gradient being from about 6 to about 2. As can be seen in Examples 2-4 and FIGS. 3-5, this method allows for a separation between an antibody comprising one VH3-chain, and an antibody comprising two VH3-chains.

According to one embodiment, the pH gradient is from about 5.5 to about 2.0, or to about 2.5. In another embodiment the pH gradient is from about 5.2 to about 2.0, or to about 2.5. In yet another embodiment, the pH gradient is from about 5.0 to about 2.0, or to about 2.5.

Within the pH range of the method, any Fc-binding of the antibody to the affinity ligand will first be released, since the Fc-binding will be abolished at a higher pH. When the Fc-binding has been neutralised, by lowering the pH, the VH-interaction is still active and will retain the mAb within the separation matrix. The affinity of the Fc-binding can still be stronger than the VH binding, but at the chosen conditions in the present method, in particular in relation to pH, the VH-interaction will remain at a lower pH. Consequently, any antibodies that do not have at least one VH3 region will be eluted prior to reaching the pH gradient range for separation. Hence, the method for separation could be used also for antibodies with Fc binding knock-out on one HC or on both HCs.

As shown in the Examples the method works well with ligands with Fc-binding still present on the affinity ligand (Examples 2-4) and an existing VH interaction. For a ligand having the VH3 interaction inhibited, no separation was observed (reference example 1).

One advantage of the method of the present invention is that the already commercially available affinity ligands may be used. The inventors have made use of an already present VH3-binding that normally is disregarded and developed a separation method based on this VH3 binding.

Antibodies suitable for separation with the present method are antibodies which are bispecific in the sense that they have one or two binding VH3 chains. It may thus relate to separation of antibodies with only one VH3 chain from antibodies with two VH3 chains. Antibodies lacking a VH3 chain may also be separated as they will be separated in the elution step from the two versions as discussed above.

The separation according to the present method results in that an antibody having one VH3-chain will elute prior to an antibody having two VH3-chains in the specified elution pH gradient. An antibody with no VH3-chain will elute prior to an antibody with one VH3-chain, since such an antibody will presumably bind the affinity ligand via the Fc portion, and consequently elute when the Fc-binding is abolished.

The affinity ligand used in the method of the present invention is preferably a protein A ligand. The protein A ligand may comprise a native or mutated domain chosen from a group comprising of Z domain, A domain, B domain, C domain, D domain and E domain. In one preferred embodiment the ligand comprises a native or mutated Z domain. In another preferred embodiment, the ligand comprises a native or mutated C domain. A separation matrix with affinity ligands that may be used in the present method may be selected from the group consisting of MabSelect™ (Cytiva), MabSelect™ Xtra (Cytiva), ProSep™ A (Merck Millipore), ProSep™ Ultra Plus (Merck Millipore), AbSolute™, CaptivA™ (Repligen), PriMab™ (Repligen) and Protein A Diamond (Bestchrom), MabSelect™ PrismA (Cytiva), Eshmuno™ A (Merck Millipore), Toyopearl™ AF-rProtein A (Tosoh), Amsphere™ A3 (JSR), and Praesto® Jetted A50 (Purolite).

The solid support of the separation matrix may be a porous support. The porous support of the separation matrix may be of any suitable well-known kind. A conventional affinity separation matrix is often of organic nature and based on polymers that expose a hydrophilic surface to the aqueous media used, i.e. exposed hydroxy (—OH), carboxy (COOH), carboxamido (—CONH₂, possibly in N-substituted forms), amino (—NH₂, possibly in substituted form), oligo- or polyethylenoxy groups on their external and, if present, also on internal surfaces.

In certain embodiments the support comprises a polyhydroxy polymer, such as a polysaccharide. Examples of polysaccharides include e.g. dextran, starch, cellulose, pullulan, agar, agarose etc. Polysaccharides are inherently hydrophilic with low degrees of nonspecific interactions, they provide a high content of reactive (activatable) hydroxyl groups and they are generally stable towards alkaline cleaning solutions used in bioprocessing.

In some embodiments the support comprises agar or agarose. The support may be commercially available products, such as crosslinked agarose beads sold under the name of Sepharose™ FF (Cytiva). In an embodiment, which is especially advantageous for large-scale separations, the support has been adapted to increase its rigidity using the methods described in U.S. Pat. No. 6,602,990 or 7,396,467 and hence renders the matrix more suitable for high flow rates.

In certain embodiments the support, such as a polymer, polysaccharide or agarose support, is crosslinked. Crosslinking is beneficial for the rigidity of the support and improves the chemical stability. In a preferred embodiment the support comprises crosslinked agarose beads.

The solid support of the separation matrix may be a convection-based chromatography matrix. Said convection-based chromatography matrix may be a fibrous substrate. Said fibrous substrate may be based on electrospun polymeric fibers or cellulose fibers, optionally non-woven fibers. The fibrous substrate may thus be a fibrous non-woven polymer matrix. The fibers are preferably crosslinked. The fibers comprised in said fibrous substrate have a cross-sectional diameter of 10-1000 nm, such as 200-800 nm, 200-400 nm or 300-400 nm. Such a fibrous substrate can be found in a HiTrap Fibro unit from Cytiva.

The feed applied to the separation matrix may be a cell culture supernatant. The cell culture supernatant may contain cells or may be depleted of cells, and/or it may be clarified.

The antibody comprised in the feed is any antibody with two different VH-chains, of which one VH-chain is VH3. Knob-into-hole (Kih) is one technology that is well validated for producing antibodies that are bispecific. Suitable bispecific antibodies for the present method may be so-called Common Light Chain Ab's. Common Light Chain Ab's have two identical light chains, and thus the bi-specificity only relates to the VH chain, which are different. Suitable bispecific antibodies may also have different light chains, in addition to different VH chains. Recently also tetra-VH IgG's have been engineered, comprising four VH chains. These tetravalent IgGs may also be separated with the present method. Duobodies is another example of a Fc-modified IgG, comprising two different VH-chains.

Antibody fragments that comprise two VH chains, of which one is a VH3 chain may also be separated by the present method. Consequently, the antibody fragment comprised in the feed may be any antibody fragment comprising two different VH-chains, of which one VH-chain is VH3. Said antibody fragment may be selected from a diabody, a scFv-Fc, a scFv-CH, Fab-scFv-Fc, or a scFv-zipper. A single-chain variable fragment (scFv) is a fusion protein of the variable regions of the heavy (VH) and light chains (VL) of immunoglobulins, connected with a short linker peptide of about 10-25 amino acids. This protein retains the specificity of the original immunoglobulin, despite removal of the constant regions and the introduction of the linker. A diabody is two scFvs with connected with linker peptides that are too short for the two variable regions to fold together (about five amino acids), forcing the scFvs to dimerize. Diabodies have been shown to have dissociation constants up to 40-fold lower than corresponding scFvs, meaning that they have a much higher affinity to their target. Two scFvs may also be connected with longer linkers, forming scFv-dimers. They may for instance be scFv-Fc or scFv-CH, or scFv-zippers.

The list of bispecific antibody fragments and bispecific antibodies above is for example only and not exhaustive. The skilled person will understand that any antibody fragment comprising at least two VH-chains, wherein one VH-chain is a VH3-chain, is applicable for the method according to the present invention.

The method of the present invention is illustrated in the non-limiting examples below. From these examples it is apparent that the method according to the claims enables a separation of a bispecific antibody from a monospecific antibody over a range of affinity ligands and separation matrices. Hence, the conceptual principle of the method of the invention is proven to be applicable to a variety of affinity ligands and separation matrices.

### EXAMPLES

Two mAbs were used in the experiments. Hemlibra® (antibody emicizumab, from Roche) is a HC heterodimer, wherein one HC has a VH3-region and one HC has a VH1-region. Herceptin® (antibody trastuzumab, from Roche) is a homodimer comprising two VH3 domains, one on each HC. In the following examples, the two mAbs were mixed before application to a chromatography column with the separation matrices specified in each example.

### Example 1 (Reference)

This experiment was performed with the antibody mix as mentioned above, using MabSelect™ SuRe separation matrix from Cytiva.

### Running Conditions:

Sample: Herceptin® and Hemlibra®, conc 1.2 and 0.6 mg/ml respectively in PBS

Column: Tricorn 5/100, Bh=10 cm, Vt=2 mL

Resin: MabSelect™ SuRe

Buffer A1: PBS

Buffer A2: 50 mM sodium citrate pH=6.5

Buffer A3: 25 mM NaOH

Buffer B1: 50 mM sodium citrate pH=2.5

System: ÄKTA™ pure

Flow: 0.5 mL/min

Equilibration: 2 CV Buffer A1

Sample appl: 200 μL

Wash 1: 2 CV Buffer A1

Wash 2: 3 CV Buffer A2

Elution: 30-100% Buffer B1 in 10 CV

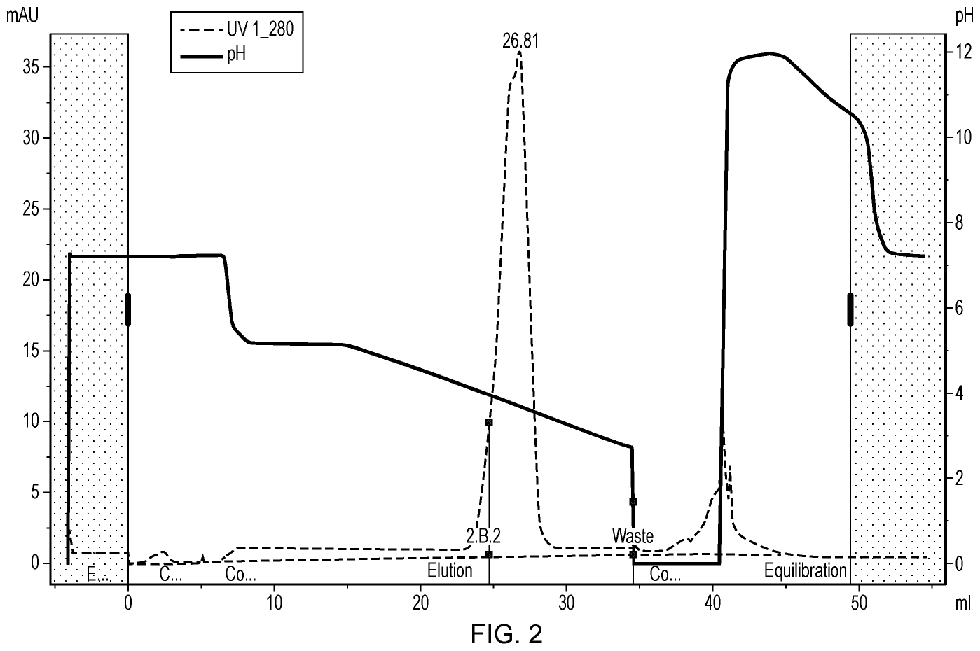

The results are shown in FIG. 2. There is no separation observed between the two mAbs. This is due to the fact that the MabSelect™ SuRe has the VH3 interaction inhibited as discussed above, with only Fc binding available on the ligand. Consequently, no separation between a bispecific antibody from a monospecific antibody, differing in their number of VH3 chains, is obtained, as the binding to the resin is based on the Fc region of the antibodies.

### Example 2

This experiment was conducted with the same mix of antibodies as above, using a PrismA separation matrix.

### Running Conditions:

Sample: Mix of Herceptin® and Hemlibra®, conc 1.2 and 0.6 mg/mL, respectively, in PBS

Column: Tricorn 5/100, Bh=10 cm, Vt=2 ml

Resin: Mabselect™ PrismA

Buffer A1: PBS

Buffer A2: 50 mM sodium citrate pH=6.5

Buffer A3: 25 mM NaOH

Buffer B1: 50 mM sodium citrate pH=2.5

System: ÄKTA™ pure

Flow: 0.5 mL/min

Equilibration: 2 CV Buffer A1

Sample appl: 200 μL

Wash 1: 2 CV Buffer A1

Wash 2: 3 CV Buffer A2

Elution: 30-100% Buffer B1 in 10 CV

The results are shown in FIG. 3. A separation between the two mAbs is observed, in a pH gradient from 5.2 to 2.5. The first elution peak around 26 ml corresponds to the Hemlibra®, and the second elution peak around 27 ml corresponds to Herceptin®.

Thus, it seems the VH3 interaction is responsible for the separation of VH1 and VH3 containing heavy chains.

### Example 3

This experiment was conducted with the same mix of antibodies as above, using a Amsphere™ A3 separation matrix from JSR.

### Running Conditions:

Sample: Mix of Herceptin® and Hemlibra®, conc 1.2 and 0.6 mg/mL, respectively, in PBS

Column: Tricorn 5/100, Bh=10 cm, Vt=2 mL

Resin: JSR Amsphere™ A3

Buffer A1: PBS

Buffer A2: 25 mM sodium citrate pH=5.5

Buffer A3: 100 mM NaOH

Buffer B1: 44 mM sodium citrate pH=2.5

System: ÄKTA™ pure

Flow: 0.5 mL/min

Equilibration: 2 CV Buffer A1

Sample appl: 100 μL

Wash 1: 2 CV Buffer A1

Wash 2: 3 CV Buffer A2

Elution: 0-100% Buffer B1 in 10 CV

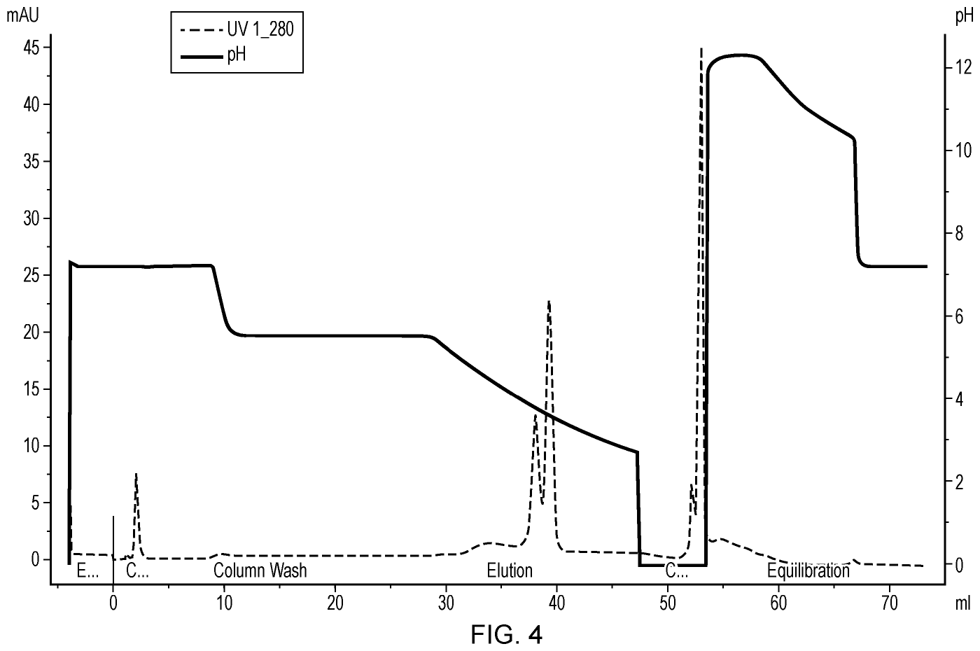

The result is shown in FIG. 4. A separation between the two mAbs is observed, in a pH gradient from 5.2 to 2.5. The first elution peak around 37 ml corresponds to the Hemlibra®, and the second elution peak around 40 ml corresponds to Herceptin.

Again, it seems the VH3 interaction is responsible for the separation of VH1 and VH3 containing heavy chains.

### Example 4

This experiment was conducted with the same mix of antibodies as above, using a Praesto® Jetted A50 separation matrix from Purolite.

### Running Conditions:

Sample: Mix of Herceptin® and Hemlibra®, conc 1.2 and 0.6 mg/mL, respectively, in PBS

Column: Tricorn 5/100, Bh=10 cm, Vt=2 mL

Resin: Purolite Praesto® Jetted A50

Buffer A1: PBS

Buffer A2: 25 mM sodium citrate pH=5.5

Buffer A3: 100 mM NaOH

Buffer B1: 44 mM sodium citrate pH=2.5

System: ÄKTA™ pure

Flow: 0.5 mL/min

Equilibration: 2 CV Buffer A1

Sample appl: 100 μL

Wash 1: 2 CV Buffer A1

Wash 2: 3 CV Buffer A2

Elution: 0-100% Buffer B1 in 10 CV

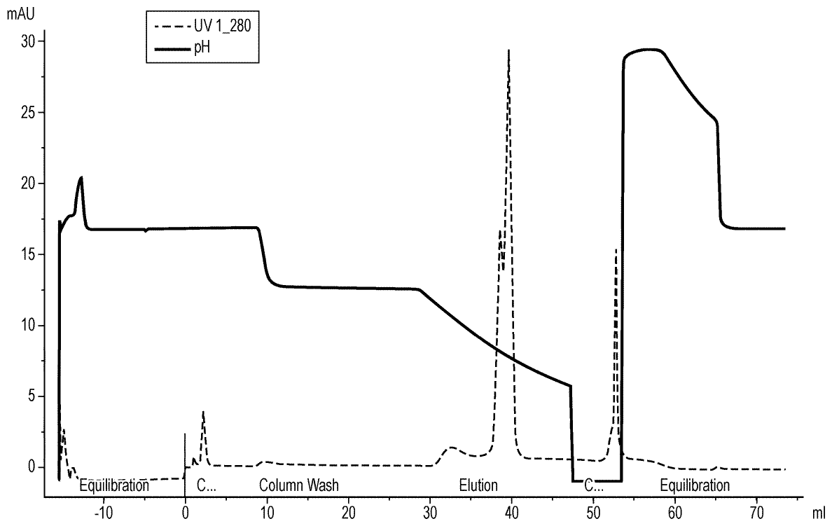

The result is shown in FIG. 5. A separation between the two mAbs is observed, in a pH gradient from 5.2 to 2.5. The first elution peak around 37 ml corresponds to the Hemlibra®, and the second elution peak around 40 ml corresponds to Herceptin®.

Once again, it seems the VH3 interaction is responsible for the separation of VH1 and VH3 containing heavy chains.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims. Any patents or patent applications mentioned in the text are hereby incorporated by reference in their entireties, as if they were individually incorporated.

1. A method of separating bispecific antibodies or bispecific antibody fragments, said method comprising the steps of:
a) providing a feed comprising bispecific antibodies or bispecific antibody fragments;
b) contacting the feed with a separation matrix having affinity ligands coupled to a support;
c) optionally washing the separation resin with a washing liquid;
d) applying an elution buffer to the separation resin, to elute the antibodies or antibody fragments bound to the affinity ligand;
wherein in step d) a pH gradient is applied over the elution buffer, said pH gradient being from about 6 to about 2.

2. The method according to claim 1, wherein the pH gradient is from about 5.5, or from about 5.2, or from about 5.0, to about 2.0, or to about 2.5.

3. The method according to claim 1, wherein the affinity ligand is a protein A ligand.

4. The method according to claim 3, wherein the protein A ligand comprises a native or mutated domain chosen from a group comprising of Z domain, A domain, B domain, C domain, D domain and E domain.

5. The method according to claim 3, wherein the protein A ligand comprises a native or mutated Z domain or a native or mutated C domain.

6. The method according to claim 1, wherein the support comprises crosslinked agarose beads.

7. The method according to claim 1, wherein the support comprises fibers with a cross-sectional diameter of 10-1000 nm, such as 200-800 nm, 200-400 nm or 300-400 nm.

8. The method according to claim 1, wherein said feed is a clarified cell culture supernatant.

9. The method according to claim 1, wherein said antibody fragment is any antibody fragment comprising two different VH-chains, of which one VH-chain is VH3.

10. The method according to claim 9, wherein said antibody fragment is selected from a diabody, a scFv-Fc, a scFv-CH, Fab-scFv-Fc, or a scFv-zipper.

11. The method according to claim 1, wherein said antibody is any antibody with two different VH-chains, of which one VH-chain is VH3.
